(19) United States
(12) Patent Application Publication
(10) Pub. No.: US 2018/0147246 A1
(43) Pub. Date: May 31, 2018

(54) Substantially Testosterone Free Compositions

(71) Applicant: Margaret Gardiner-Hunt, Doylestown, PA (US)

(72) Inventor: Margaret Gardiner-Hunt, Doylestown, PA (US)

(21) Appl. No.: 15/365,830

(22) Filed: Nov. 30, 2016

(51) Int. Cl. A61K 36/185; A61K 9/00; A61K 47/10; A61P 5/26

(52) U.S. Cl. CPC A61K 36/185; A61P 5/26; A61K 47/10; A61K 9/0014

(57) ABSTRACT

1. A therapeutic composition, substantially free of testosterone, comprises
(a) at least one Bulbine natalensis compound present in a therapeutically effective amount to increase testosterone levels when administered to a mammal; and
(b) a pharmaceutical carrier effective for the delivery of said Bulbine natalensis compound in a form of one suitable for an oral or topical administration

This application is a full utility patent application of U.S. Ser. No. 62/260,756 that was filed on Nov. 30, 2015.

### FIELD OF THE INVENTION

The present invention relates to topical formulations for the treatment of hormonal disorders and diseases that are caused by and/or related to low levels or the absence of testosterone in a mammal. More specifically, said compositions comprise therapeutically effective amounts of a plant extract from Bulbine natalensis in a pharmaceutical carrier for the prevention and treatment of disorders and diseases associated with testosterone insufficiency and deficiency.

### BACKGROUND OF THE INVENTION

Testosterone is a steroid hormone that has been synthesized from cholesterol since 1935. Though present in small amounts in females, the hormone in men determines men's sexuality, physical development and as such is considered the male sex hormone. Low testosterone levels in men can have adverse effects such as reduced libido, decreased muscle mass and strength, erectile dysfunction, loss of body hair, gynecomastia (development of abnormal breast enlargement), underdeveloped testes, hypotrophic testes, less stamina, fine facial wrinkles and a worsened sense of well being.

Testosterone plays a key role in the development of male reproductive tissues as well as promoting predominantly male characteristics such as increased muscle, bone mass, and the growth of body hair. Moreover, the level of testosterone in men has been shown to decrease with increasing age. Total testosterone levels are said to decline at a rate of 1.6% per year, and levels of bioavailable testosterone are reduced by approximately 2.0-3.0% per year according to the results obtained from the Massachusetts Male Aging Study. (Feldman et al., 2002).

Testosterone insufficiency (also termed hypo-testosteronism or hypotestosteronemia) is a condition due to abnormally low testosterone production. It may occur because of testicular dysfunction (primary hypogonadism) or hypothalamic-pituitary dysfunction (secondary hypogonadism) and may be congenital or acquired. Doctors have for a long time wondered about the existence of male menopause.

While various formulations and methods have been developed as a replacement therapy for men due to disorders of the testicles, pituitary gland, or brain that cause hypogonadism or other conditions are known in the art, these formulations require the presence of testosterone or a synthetic testosterone made by esterification or alkylation or the incorporation of testosterone analogs such as fluoxymesterone and methyltestosterone

It is therefore an object of the present invention to provide formulations that include therapeutically effective amounts of non-steroidal compositions, particularly substantially free of testosterone in its preparation, for the prevention and treatment of disorders and diseases associated with testosterone insufficiency and deficiency. It is a further object of the present invention to provide therapeutically effective amounts of non-steroidal compositions, particularly substantially free of testosterone, for the prevention and treatment of disorders and diseases associated with testosterone insufficiency and deficiency, that may be readily applied to, and absorbed by the skin.

### Abstract of the Invention

1. A therapeutic composition, substantially free of testosterone, comprises
(a) at least one Bulbine natalensis compound present in a therapeutically effective amount to increase testosterone levels when administered to a mammal; and
(b) a pharmaceutical carrier effective for the delivery of said Bulbine natalensis compound in the form of one suitable for an oral or topical administration

### DETAILED DESCRIPTION OF THE INVENTION

The present invention is directed to non-hormonal cosmetic and pharmaceutical formulations/compositions substantially testosterone-free in therapeutically effective amounts, formulated in a pharmaceutical carrier for the prevention and treatment of disorders and diseases associated with low levels of testosterone. These inventive formulations include a plant extract, Bulbine natalensis in specific amounts to replace and prevent testosterone deficiency and/or testosterone insufficiency in mammals. The inventive formulations include Bulbine natalensis in specific therapeutic amounts for treating testosterone deficiency and/or testosterone insufficiency in mammals.

In certain embodiments of the present invention, Bulbine natalensis extract is provided in specific therapeutic amounts for preventing and/or treating diseases associated with testosterone deficiency and/or testosterone insufficiency. These include, for example, diseases and conditions associated with sexual drive including erectile dysfunction, infertility, delayed puberty, fragile bones including those conditions caused by osteoporosis, osteopenia, osteomalacia, decreases in muscle mass, increase in body fat, gynaecomastia, decreased sperm count, decreases in hemoglobin (mild anemia), the loss of body hair, changes in blood serum cholesterol and/or lipid levels, insulin resistance, coronary artery disease, depression, fatigue, the loss of one's ability to concentrate and wrinkled skin.

Bulbine natalensis is a plant from the family of Asphodelaceae that has been reported to possess aphrodisiac properties. The leaf sap is also used for topical purposes such as cracked lips and burns while the aqueous extract of the leaves is used for intestinal issues (diarhhea, vomiting, convulsions) and has been reported to be used for anti-diabetic and anti-rheumatoid purposes. It has been used by various cultures around where it grows (Northern and Eastern sections of Africa) and is called ingcelwane in Xhosa, rooiwortel in Afrikaans, and ibhucu in Zulu.^{[2]}

As an oral compound, the Bulbine natalensis extract of the present invention may contain a variety of isolated compounds or classes of compounds. These include:
Saponins at 1.97% of dry weight
Anthraquinones at 0.152% dry weight
Tannins at 0.481% dry weight
Cardiac Glycosides at 0.887% dry weight
Alkaloids at 0.2% dry weight

Unless otherwise defined herein, scientific and technical terms used in connection with the present invention shall have the meanings that are commonly used and understood by those of ordinary skill in the art. The meaning and scope of the terms should be clear, however, and in the event of any limitation, the Bulbine natalensis extract may be provided in its forms as Bulbine natalensis, Bulbine natalensis anthericum latifolium L.f., Bulbine natalensis baker, Bulbine natalensis baker stem extract, broad-leaved bulbine natalensis, their precursers, inactive forms, active forms and the metabolites thereof.

U.S. Pat. No. 9,180,158 to Widgerow et al teaches the incorporation of a Bulbine frutescens extract in a topical composition for treating damaged skin of a human in need thereof consisting essentially of an extract of the compound in an amount from about 9% to about 11% mass per mass of the total topical composition, together with a Centella asiatica extract in an amount from about 0.1% to about 2.0% mass of the composition, an olive extract as well as other excipients and stabilizers. The composition is disposed onto a tape which is topically applied onto the skin of a human in need thereof.

U.S. Pat. No. 8,486,459 to Colson et. al. discloses a method of producing a Bulbine frutescens extract in a stable form by treating juice expressed from the leaves of the Bulbine frutescens plant with hydrogen peroxide, removing the remaining hydrogen peroxide and stabilizing the extract using a suitable stabilizer. It is disclosed that the Bulbine frutescens extract is disclosed as being useful in the dermal therapy or cosmetic fields and has an especially good effect on diseases of the skin and mucous membranes. The teachings and disclosure of the Colson '459 patent and the extraction methodology are hereby incorporated by reference. In several embodiments, the topical composition is in the form of a gel, cream or ointment. In one embodiment, the topical composition further comprises at least one ingredient selected from the group consisting of water, a solvent, a preservative, a surfactant, a gelling agent, and a pH balancer. In one embodiment, all of these ingredients are combined with Bulbine Centella, and olive extracts. The claimed topical composition further comprises at least one ingredient selected from the group consisting of phospholipids, amino acids, vitamins and peptides, and phosphatidylserine

Despite its' known uses in the literature and prior art, it has been surprisingly and unexpectedly discovered that a therapeutically-effective amount of Bulbine natalensis extract can be incorporated into a substantially testosterone-free formulation and increase testosterone levels in mammals. More specifically, the present invention has found that the Bulbine natalensis formulations are readily absorbed into the bloodstream by oral ingestion and through topical and transdermal application to the skin whereby therapeutically-effective amounts of Bulbine natalensis extract results in rapid biological increases in testosterone in mammals substantially free of testosterone in the inventive formulations of the present invention.

More specifically, a therapeutically-effective amount of the Bulbine natalensis extract in the present invention can be applied as a topical transdermal formulation to the skin of a mammal, and result in an increase in the serum blood levels of testosterone in said mammal, substantially free of testosterone in the inventive formulations with a reduced risk of “testosterone” transfer to other mammals. As the inventive formulations do not contain testosterone, the serious risk of testosterone exposure to women, children and pets who live or interact closely with individuals who are using products containing testosterone is significantly reduced. It is known in the art that a testosterone transfer can occur from physical contact with testosterone, from the application site, unwashed clothes or linens or any area such as a sink or counter that may have come in contact with the testosterone. Also as is known in the art, topical and transdermal delivery systems are not subjected to the challenges of oral absorption (e.g. breakdown and diminished absorption in the stomach and small intestine wherein much of the actives' potency may be degraded and lost). Absorption through the skin thereby avoiding the gastrointestinal track improves bioavailability of ingredients and hence improves therapeutic results.

As used herein, a “therapeutically effective amount” refers to an amount of the Bulbine natalensis extract that is effective to achieve a desired therapeutic result at a particular dosage and over a particular period of time. The amount may vary based on the degree of the individuals' condition, the individuals' age, sex, weight, health, metabolic rate and the individuals tolerability, side effects and/or toxicity resulting from administration.

Moreover and more specifically, as used herein, a “therapeutically effective” amount refers to an amount of B. natalensis extract that is absorbed into the skin over a period of time to cause a measurable increase in blood serum total testosterone, blood serum free testosterone and/or blood serum bio-available testosterone, which may be determined through the use of conventional blood tests, pharmaco-kinetic analyses and techniques known to those of skill in the art. The formulations of the present invention may be applied to the skin on different areas of the body and without limitation, the concentration of Bulbine natalensis, the delivery carrier and the inclusion of specific ingredients such as penetration enhancers, stabilizers and so forth may be employed. In certain embodiments, it may be intended that the inventive formulations be re-applied after a certain period of time for a particular therapeutic purpose, which will be taken into account in determining the concentration of the B. natalensis and the other ingredients in such formulations.

A “therapeutically effective amount” of Bulbine natalensis extract that is present in the formulations of the present invention is an amount that improves one or more conditions, metabolic disorders and/or disease states associated with low blood serum levels of and/or testosterone insufficiency and/or testosterone deficiency in a mammal, including those discussed herein, regardless of whether such testosterone deficiency and/or testosterone insufficiency is due to chemical, environmental, psychological, physical, nutritional and/or physiological factors.

By way of further example and without limitation, a “therapeutically effective amount” of B. natalensis extract that is present in the formulations of the present invention is one in which a specific amount of the extract is administered systemically to a mammal. The extract will elicit the natural production of testosterone that is unable to be produced within the mammals' body. It will be recognized and understood by those of ordinary skill in the art, that based on the teachings herein, one will be capable of empirically determining the therapeutically effective amount of B. natalensis extract needed in specific formulations which are embodiments of the present invention, to achieve a particular therapeutic benefit without the need for undue experimentation. It will also be possible to determine the therapeutically effective amount of other agents that may be included in the inventive formulations in combination with the Bulbine natalensis extract to provide additional therapeutic benefits.

Preferably, the inventive formulations of the present invention may include the following delivery systems for the extract, in forms suitable for topical administration thereof. These provide the transdermal delivery of a therapeutically effective amount of Bulbine natalensis extract in the form of a cream, gel, liquid, lotion, ointment, solution, spray emulsions, aerosol, and combinations thereof. These may include multi-lamellar vesicles, various lipid structures including liposomes, nano-spheres, microsponges, or combinations thereof. In certain exemplary, non-limiting embodiments, the Bulbine natalensis extract may be encapsulated in the formulation and later released when ruptured for a delayed time release of the testosterone—stimulating compound. Encapsulating materials and techniques for the compounds encapsulation, including time-release encapsulation, are well known in the art.

Other conventional cosmetic and/or pharmaceutical agents may be provided in the formulations of the present invention, so long as they are non-toxic and physiologically acceptable and suitable for use in combination with the therapeutically effective amount of Bulbine natalensis provided in the formulations. For example, the claimed formulations of the present invention may include chemically compatible pharmaceutical vehicles and excipients such as water and/or alcohol. The inventive formulations may also include emollients, such as petrolatum, zinc oxide, paraffin, minerol oil, glycerin, beeswax, olive oil, coconut oil, jojoba oil, lanolin, cocoa butter, butyl stearate, stearic acid, di-glycol laurate, 2-ethylhexanol, almond butter, aloe vera gel, batana oil, caprylic/capric triglycride, caprylyl-caprylate/caprate, cetyl palmitate, chia seed oil, coco-caprylate, collodion, dhupa butter, dicaprylyl carbonate, dihydroxyacetone, dimetheicone, myristates, shea butter, plant oils, fatty acids, fatty alcohols, triglycerides, benzoates, palmitates, squalene and ceramides, derivatives, combinations and mixtures thereof. The compositions may include skin conditioning agents, such as butyl alcohol, cholesterol, lanolin, fatty acid esters, fatty acid ethers, cetyl acetate, silicones, plant oils, panthenol, panthenol triacetate, vitamin B, vitamin C, vitamin D, vitamin E, vitamin D, keratin, lysine, arginine, hydrolyzed wheat proteins, hydrolyzed silk proteins, colloidal oatmeal, zinc, coal tar, hydrocortisone, sulfides, emollients, derivatives, combinations and mixtures thereof.

The inventive formulations may include pH adjusting agent(s), such as alpha—hydroxy acids, buytylated hydroxy toluene (BHT), ethylene diamine tetra—acetic acid (EDTA), triethanolamine (TEA), cosmetics salts, glycerine, propylene glycol and derivatives, combinations and mixtures thereof.

Other acceptable ingredients include humectants, such as glycerine, propylene glycol, sorbitol, hexylene glycol, butylene glycol, urea, alpha-hydroxy acids, polyhydric alcohols, sorbital, hydroxypropyl sobitol, hexylene glycol, 1-3 dibutylene glycol, 1,2,6-hexanetriol, ethoxylated glycerol, propoxylated glycerol, and derivatives, combinations and mixtures thereof may be incorporated herein. Pharmaceutical acceptable buffering agents, such as citric acid, sodium citrate, their derivatives, combinations and mixtures thereof are also useful beneficial.

Other components that may be included are viscosity adjusting agents, such as salts, carbomer gelling agents, gum derivatives, and derivatives, combinations and mixtures thereof. Preservatives, such as methylparaben, ethylparaben, butylparaben, formaldehyde, DMDM hydantoin, leucidal liquid, propylparaben, phenooxyethanol and derivatives, combinations, and mixtures thereof are also useful in small amounts. The inventive formulations may include emulsifying agents, such as polysorbate 80, glyceryl disterate, POE (2) stearyl ether, POE 10 stearyl ether, ceateareth 20, stearyl alcohol, ceteareth 20, cetearyl alcohol, lecithin and derivatives, combinations and mixtures thereof. For example, the inventive formulations my include chelating agents such as ethylenediamine tetra acetic acid (EDTA), dihydroxyethyl glycine, tartaric acid, derivatives, combination, and mixtures thereof. These formulations may also include thickening agents, such as salt, silica, bentonite, magnesium aluminum silicate, carbomer, gum, xanthan gum, gelatin, cetyl alcohol, stearyl alcohol, carnauba wax, stearic acid, polyacrylamide, C13-C14 isoparafin, laureth-, and derivatives, combinations and mixtures thereof.

Additional optional elements of the formulations of the present invention include anti-oxidants, such as green tea extract, ascorbyl palmitate, tocopheryl acetate, BHT, BHA, alpha lipoic acid, beta-glucan, coenzyme Q10, grape seed extract, green tea, soybean sterols, superoxide dismutase, vitamin C (ascorbyl palmitate and magnesium ascorbyl palmitate), and vitamin E (alpha tocopherol, tocotrienols, tocopherol acetate), pomegranate, curcurmin, turmeric, butylated hydroanisole (BHA), phenyl-\dot{a}-naphthylamine, hydroquinone, propyl gallate, nordihydroquiaretic acid, and derivatives, combinations and mixtures thereof.

The inventive formulations of the present invention may include nutrients and amino acids such as alanine, arginine, asparagine, aspartic acid, cysteine, cystine, glutamic acid, glutamine, glycine, histidine, isoleucine, leucine, lysine, methionine, phenylalanine, proline, serine, threonine, tryptophan, tyrosine, valine, minerals and vitamin A, vitamin B, vitamin C, vitamin D, vitamin E, vitamin K, and derivatives, combinations, and mixtures thereof. the inventive formulations may include fragrances, such as eucalyptus oil, camphor synthetic, peppermint oil, clove oil, olive oil, lavender, chamomile, flavor fragrances such as chocolate, vanilla, mint, derivatives, combinations and mixtures thereof. The formulations of present invention may also include other food additives commonly contained in supplements such as gelatin, rice, flour, wheat, citric acid, natural and artificial flavors, derivatives, combinations and mixtures thereof.

The inventive formulations may also include colorants, such as zinc oxide (white), titanium dioxide (white), blue, green, orange, red, violet, yellow and black. The inventive formulations may also include mixtures and combinations of any of the above, as well as one or more active ingredients in addition to the therapeutically effective dose of B. natalensis, for example and without limitation, other therapeutic hormonal agents for the prevention and treatment of one or more conditions or disease states associated with testosterone deficiency or testosterone insufficiency.

The inventive formulations of the present invention may also include sun protecting ingredients with an SPF of 6-95. Preferably, the bulbine natalensis extract is present in the inventive formulations of the present invention at concentrations of from about 0.1% to about 60.0% More preferably, the Bulbine natalensis extract is present in the formulations at concentrations of from about 0.1% to about 80.0%. Most preferably, the B. natalensis extract is present in the inventive formulations of the present invention at concentrations of from about 1.0% to about 50.0% for topical and transdermal applications and at concentrations of from about 50% to 98% for oral formulations. In certain exemplary, non-limiting embodiments, the inventive formulations result in increases in testosterone levels of 1.0% or greater depending on the therapeutic amount and the number of days of therapy.

The Bulbine natalensis compound is present in the compositions of the present invention in a therapeutically-effective amount to increase said mammal's biological testosterone levels. That being said, generally the therapeutic compositions of the present invention are formulated to account for the age of the mammal, the pre-treatment testosterone levels of the mammal and the desired therapeutic effect.

The following examples are provided herein to more specifically set forth the various compositions and methods of the present invention. It is recognized that changes may be made to the specific components and ranges disclosed herein, and that there are a number of ways known in the art to change the disclosed variables. That being said, it is to be understood that the exemplary embodiments disclosed herein, however, are understood to be illustrative and the invention should not be so limited and should be construed in terms of the spirit and scope of the claims that follow.

### Example 1

Preparation of testosterone—free formulations for treating testosterone deficiency and/or testosterone insufficiency and for the prevention of disorders and disease states associated therewith. The topical testosterone-free, Bulbine natalensis formulation of the present invention was prepared as a viscous gel or lotion comprising the following ingredients.

Bulbine natalensis extract was mixed with 8.0 mLs. propylene glycol, and a thickening agent, (acrylic acid/alkyl methacrylate-co-enhancer 5.0-1.5 copolymer Pemulen-TRI). thickening agent ethanol, 200 proof), USP-solvent 73.6 glycerin, USP-co-solvent, emollient, humectant, and protein 5.0 stabilizer polyethylene glycol 1000, NF-crystallization inhibitor 0.5M tris amino crystal-neutralizing agent 0.1 water, sterile, for irrigation, USP 5. wherein amount of B. natalensis was varied in amount according to the weight and size of the individual in need thereof.

The ingredients were weighed and mixed in a vessel which was closed with a stopper to prevent evaporation. Oxacyclohexadecan-2-one, ethanol, propylene glycol, and glycerin were then weighed and mixed in a bottle beaker. The mixing was carried out at approximately 22° C. Eighty (80) grams of Bulbine natalensis extract in solid form were warmed at approx 45° C. in a water bath until molten and added to a vessel. Four-hundred grams of ethanol were then added to the vessel while using portions to repeatedly rinse out the bottle beaker which contained the plant extract. Fifty (50) gms of propylene glycol and fifty gms of glycerin were then added separately to the vessel and the resulting mixture was stirred gently. Ten (10) additional grams of Bulbine natalensis powder were then added to the vessel and the resulting mixture was stirred until the solids were dissolved completely. Five (5.0) grams of polyethylene glycol were then added to the vessel and the resulting mixture was stirred until the polyethylene glycol was dissolved. Three (3.0) grams of acrylic acid/alkyl methacrylate copolymer and 15 grams of carboxypolymethylene were then added separately, in that order, to the vessel and the resulting mixture was then stirred for approximately one hour and twenty minutes. Three hundred and thirty-six (306) grams of ethanol were added to the vessel. While stirring the contents of the vessel, 50 grams of water and 1 gram of tris—amino crystal were combined and weighed in one of the previously used bottle beakers, shaken until dissolved, and slowly added drop-wise over 20 minutes to the center of the vessel. Stirring of the resulting mixture continued for approximately 18 hours. A colorless, clear to translucent gel was recovered with a viscosity of about 3,500 cps and a musk-like fragrance. The gel is capable of being squeezed or pumped from a suitable container vessel known in the art.

### Example 2

A composition comprising a mixture of Bulbine natalensis extract powder combined with flour, magnesium stearate, silicone dioxide and titanium dioxide. The ingredients are mixed together in a vessel until the ingredients are thoroughly mixed. The combined ingredients are then filled into a gelatin capsule.

### Example 3

A composition comprising 3.0 wt. % Bulbine natalensis extract; 1.0 wt. % of a skin penetration enhancer (oxacyclohexadecan-2-one); 1.0 wt. % propylene glycol, a co-enhancer 5.0 carboxypolymethylene; 1.0 wt. % acrylic acid/alkyl methacrylate copolymer (thickening agent); 0.5 wt. % also a thickening agent; 200 proof ethanol (EtOH), USP-solvent 69.6 glycerin, USP co-solvent, emollient, humectant, and protein 5.0 stabilizer polyethylene glycol 400, NF-crystallization inhibitor 0.5 tris amino crystal-neutralizing agent 0.08 water, sterile, for irrigation, USP 16.32 was prepared in a lot size of 400 grams. All ingredients were weighed accurately. Oxacyclohexadecan-2-one, ethanol, propylene glycol, and glycerin were weighed in a bottle beaker. The ingredients were mixed at ambient temperature between each step of ingredient addition.

The resulting Bulbine natalensis extract paste is therapeutically effective to raise the low testosterone levels in testosterone deficient individuals, thereby resulting in increased testosterone levels in such individual, which may thereby prevent the onset of various disease states associated with low teasterone levels. And as with the treatment formulations discussed above, these treatment formulations may be derived in which the concentration of B. natalensis extract and other ingredients are selected for each formulation based on the individual's profile (e.g. age, weight, health, current testosterone levels, etc.) and the individual's testosterone therapeutic goal (e.g. to gradually improve testosterone levels or to rapidly treat low testosterone). These considerations include whether the formulation is dosed once a day or is intended to be dosed more than once daily, if the formulation is ingested or applied topically, as well as the specific metabolic and absorption characteristics of the individual that may affect the delivery rate of the B. natalensis extract. Accordingly, formulations according to this example may be provided for use by a number of individuals engaged in varied activities and using these treatment formulations under varied conditions, while in all cases in the inventive formulations substantially free of testosterone while providing an increase in biological testosterone in such individuals.

The dermal B. natalensis composition is prepared according to dermal formulation art-recognized techniques known in the art in order to have a therapeutically effective amount of the compound or ingredient which can replace or increase the biological testosterone in the body so as to achieve a particular therapeutic benefit and reduce the risk of medical conditions, disorders and diseases associated with low testosterone. In such composition or formulation, the amount of Bulbine natalensis will depend on a number of factors, including the desired dose of the resulting formulation. For example, a lower dose may be desired to maintain testosterone levels, while testosterone insufficient or deficient mammals (those completely lacking blood serum testerone) may require a composition or formulation with a higher therapeutic dose. In all cases, however, the inventive formulation provides a therapeutically effective amount of bulbine natalensis to replace or increase testosterone.

These inventive treatments and/or preventative methods may be provided with different concentrations or amounts of the Bulbine natalensis extract and the other ingredients which are selected may be varied and tailored according to the surrounding environmental and other conditions in which the inventive composition or formulation is intended to be used. For example, whether the inventive composition or formulation is intended to be re-dosed or re-applied and/or re-dosed or re-applied after a specific period of time or activity, and also taking into account specific characteristics of the individual for whom it is intended that may impact the absorption of the extract by the individual. In such composition or formulation, the choice of bulbine natalensis will depend upon a number of factors, including the desired dose of the resulting formulation. For example maintaining, a testosterone serum level differs by age group as evidenced by the total testosterone reference for males:

| Age | Amount | |
|---|---|---|
| 0-5 months: | 75-400 | ng/dL |
| 6 months-9 years: | <7-20 | ng/dL |
| 10-11 years: | <7-130 | ng/dL. |
| 12-13 years: | <7-800 | ng/dL |
| 14 years: | <7-1,200 | ng/dL |
| 15-16 years: | 100-1,200 | ng/dL |
| 17-18 years: | 300-1,200 | ng/dL |
| > or = 19 years: | 240-950 | ng/dL |

### Example 3

Testosterone free formulations for treating disorders and disease states associated testosterone deficiency and/or testosterone insufficiency.

A testosterone-free formulation was prepared according to procedures well known in the art that will yield an amount of B. natalensis extract that is therapeutically effective to treat a disorder and/or disease state associated with low blood serum levels of testosterone. As discussed herein, such disorders and/or disease states which may be treated using the inventive formulations, include for example and without limitation, conditions, disorders and diseases associated with sexual function including erectile dysfunction, infertility, delayed puberty, fragile bones including osteoporosis, osteopenia, osteomalacia, decreases in muscle mass, increase in body fat, gynaecomastia, decreased sperm count, decreases in hemoglobin (mild anemia), decrease in body hair, changes in cholesterol and/or lipid levels, insulin resistance, coronary artery disease, depression, fatigue, loss of concentration and wrinkled skin.

In all cases, these formulations provide a therapeutically effective amount of bulbine natalensis that may be is administered orally or transdermally to increase testosterone levels, and desirably to optimal testosterone levels substantially free of testosterone in the formulation. With the treatment formulations discussed above, these treatment formulations may be provided in which concentration of bulbine natalensis and other ingredients are selected for each formulation based on specific intended use of the resulting formulation, including the delivery (take orally by pills/capsules, drops, syrups, mucus membrane deliveries or applied topically and where it is applied to the body (testes, skin, mucus membranes, etc), whether the formulation is intended to be re-dosed or re-applied after particular activities or after specific periods of time, and specific characteristics of the individual that may impact the absorption of bulbine natalensis formulations.

Accordingly, formulations according to this example may be provided for use by a number of individuals engaged in varied activities and using these treatment formulations under varied conditions, while in all case substantially free of testosterone while increasing testosterone levels in such individuals.

From the teachings provided herein, those of ordinary skill in the art will be able to make formulations having a therapeutically effective amount of Bulbine natalensis extract, and moreover, the safety and efficacy of such formulations may be tested and modified in established animal models using conventional pharmacokinetic analysis and techniques. These formulations can also be prepared using the described ingredients to render them suitable for use by specific individuals according to their need and metabolic activity, and/or for using during particular activities, and/or for use on particular areas of the body, and/or for use when exposed to particular environmental conditions. In such composition or formulation, the choice of B. natalensis will depend on a number of factors, including the desired dose of the resulting formulation. For example, a lower dose may be desired to maintain testosterone levels, while testosterone insufficient or deficient mammals may require a composition or formulation with a higher therapeutic dose. In all cases, however, the inventive formulation provides a therapeutically effective amount of bulbine natalensis that is administered topically with further enhancement of testosterone.

As with inventive treatments, these inventive treatments and/or preventative methods may be provided with different concentrations of B. natalensis and other ingredients which are selected for each inventive composition or formulation, including the environmental and other conditions in which the inventive composition or formulation is intended to be used, as whether the inventive composition or formulation is intended to be re-applied after particular activities and/or specific period of time, and also taking into account specific characteristics of the mammal for whom it is intended that may impact absorption of Bulbine natalensis in such mammal.

What is claimed is:

1. A therapeutic composition, in the absence of testosterone, comprising
(a) at least one bulbine natalensis compound present in a therapeutically effective amount to increase testosterone levels when administered to a mammal; and
(b) a topical transdermal pharmaceutical carrier effective for the therapeutic delivery of said bulbine natalensis

2. The composition according to claim 1, wherein said bulbine natalensis compound is present in an amount sufficient to compensate for a reduction in said mammal's natural testosterone production and/or said mammal's blood serum testosterone level.

3. The composition according to claim 1, wherein said mammal has low, insufficient or deficient testosterone levels, and wherein the topical administration of said composition to said mammal results in an increase in testosterone levels in said mammal.

4. (canceled)

5. The composition according to claim 3, wherein a mammal with testosterone deficiency has a testosterone blood serum level of less than 300 ng/dl or a mammal with testosterone insufficiency has a testosterone blood serum level in the range of about 300 ng/dl to about 400 ng/dl or a mammal with low testosterone which is a testosterone blood serum level lower than normal for said mammal.

6. The composition according to claim 1, wherein said topical transdermal pharmaceutical carrier results in transdermal delivery of the bulbine natalensis in therapeutic amounts into the skin in the absence of irritating penetration enhancers such as alcohols, glycols, DMSO (dimethyl sulfoxides), or terpenes.

7. The composition according to claim 1, wherein said bulbine natalensis compound is selected from the group consisting of bulbine natalensis, anthericum latifolium L.f., bulbine natalensis baker, bulbine natalensis baker stem extract, broad-leaved bulbine, extracts, precursers, inactive form, active forms and metabolites thereof.

8. The composition according to claim 1, further comprising at least one emollient wherein at least one emollient is selected from the group consisting of such as petrolatum, zinc oxide, paraffin, mineral oil, medium chain triglycerides, glycerin, beeswax, olive oil, coconut oil, jojoba oil, lanolin, cosmetic butters including cocoa butter, butyl stearate, stearic acid, diglycol laurate, 2-ethylhexanol, almond butter, aloe vera gel, batana oil, caprylic/capric triglycride, caprylyl-caprylate/caprate, cetyl palmitate, chia seed oil, coco-caprylate, dihydroxyacetone, dimetheicone, myristates, shea butter, plant oils, fatty acids, triglycerides, benzoates, palmitates, squalene and ceramides, derivatives, combinations and mixtures thereof and/or a skin condition agent wherein said skin condition agent is selected from the group consisting of such as cholesterol, lanolin, fatty acid esthers, cetyl acetate, silicones, plant oils, panthenol, panthenol triacetate, vitamin B, vitamin C, vitamin D, Vitamin E, vitamin D, keratin, lysine, arginine, hydrolyzed what proteins, hydrolyzed silk proteins, colloidal oatmean, zinc, coal tar, hydrocortisone, sulfides, emollients, derivatives, combinations and mixtures thereof and/or a humectant, wherein said humectant is selected from the group consisting of such as glycerine, urea, alpha-hydroxy acids, sorbital, and derivatives, combinations and mixtures thereof or an emulsifying agent, wherein said emulsifying agent is selected from the group consisting of polysorbitate 80, glyceryl disterate, POE (2) stearyl ether, POE 10 stearyl ether, ceateareth 20, ceteareth 20, cetearyl alcohol, lecithin and derivatives, combinations and mixtures thereof and/or a preservative, wherein at least one preservative is selected from the group consisting of methylparaben, ethylparaben, butylparaben, formaldehyde, DMDM hydantoin, propylparaben, phenyloxyethanol, Lactobacillus Ferment & Lactobacillus & Cocos Nucifera (Coconut) Fruit Extract and derivatives, combinations, and mixtures thereof and/or a pH adjusting agent wherein said pH adjusting agent is selected from the group consisting of alpha hydroxy acids, bytylated hydroxy toluene (BHT), ethylene diamine tetra acetic acid (EDTA), triethanolamine (TEA), cosmetics, salts, glycerine, propylene glycol and derivatives, combinations and mixtures thereof, and/or a buffering agent, wherein said buffering agent is selected from the group consisting of citric acid, sodium citrate, and derivatives, combinations and mixtures thereof, and/or a viscosity adjusting agent, wherein said viscosity adjusting agent is selected from the group consisting of salt, carbomer gelling agents, gum derivatives, and derivatives, combinations and mixtures thereof and/or a chelating agent, wherein said at least one chelating agent is selected from the group consisting of ethylenediamine tetra acetic acid (EDTA), dihydroxyethyl glycine, tartaric acid, derivatives, combination, and mixtures thereof and/or a thickening agent, wherein said at least one thickening agent is selected from the group consisting of salt, silica, bentonite, magnesium aluminum silicate, carbomer, gum, xanthan gum, gelatin, cetyl alcohol, stearyl alcohol, carnauba wax, stearic acid, polyacrylamide, C₁₃-C₁₄ isoparafin, laureth-, and derivatives, combinations and mixtures and/or an antioxidant, wherein said antioxidant is selected from the group consisting of green tea extract, ascorbyl palmitate, tocopheryl acetate, BHT, BHA, alpha lipoic acid, beta-glucan, coenzyme 010, grape seed extract, green tea, soybean sterols, superoxide dismutase, vitamin C (ascorbyl palmitate and magnesium ascorbyl palmitate), and vitamin E (alpha tocopherol, tocotrienols, tocopherol acetate), pomegranate, curcurmin, turmeric, butylated hydroanisole (BHA), phenyl-a-naphthylamine, hydroquinone, propyl gallate, nordihydroquiaretic acid, and derivatives, combinations and mixtures thereof, and/or a nutrient is selected from the group consisting of alanine, arginine, asparagine, aspartic acid, cysteine, cystine, glutamic acid, glutamine, glycine, histidine, isoleucine, leucine, lysine, methionine, phenylalanine, praline, serine, threonine, tryptophan, tyrosine, valine, minerals and vitamin A, vitamin B, vitamin C, vitamin D, vitamin E, vitamin K, and derivatives, combinations, and mixtures thereof and/or a fragrance, wherein said at least one fragrance is selected from the group consisting of eucalyptus oil, camphor synthetic, peppermint oil, clove oil, olive oil, lavender, chamomile, flavor fragrances such as chocolate, vanilla, mint, derivatives, combinations and mixtures thereof and/or a colorant, wherein said colorant is selected from the group zinc oxide (white), titanium dioxide (white), blue, green, orange, red, violet, yellow and black.

9. The composition according to claim 1, wherein said bulbine natalensis compound and said topical transdermal pharmaceutical carrier are provided in the form of a cream, gel, ointment, liquid, lotion, spray, emulsion, aerosol or combination thereof.

10. The composition according to claim 1, wherein said mammal has at least one condition, disorder or disease associated with low testosterone, testosterone deficiency or testosterone insufficiency.

11. The composition according to claim 10, wherein said at least one condition, disorder or disease state associated with low testosterone, testosterone deficiency or testosterone insufficiency is selected from the group consisting of conditions, disorders and diseases associated with sexual function, bone-related disorders, physical changes to the body, metabolic disorders, coronary artery disease, emotional disturbances and wrinkled skin.

12. The composition according to 11, wherein said sexual function-related disorders and diseases are selected from the group consisting of erectile dysfunction, premature ejaculation, decreased sperm count, infertility, delayed puberty, impaired sexual drive and low libido.

13. (canceled)

14. The composition according to claim 11, wherein said physical changes to the body are selected from the group consisting of decreased muscle mass/strength, increase in body fat, gynaecomastia, breast swelling/tenderness, hot flashes, sleep disturbances, fatigue, decreases in hemoglobin (mild anemia), decrease in body hair and metabolic disorders to the body are selected from the group consisting of changes in cholesterol and/or lipid levels and/or insulin resistance.

15. The composition according to 11, wherein said emotional disturbances are selected from the group consisting of sadness, depression, overall decreased sense of well-being, reduced memory and concentration, and lowered motivation and self-confidence.

16. The composition according to claim 1, wherein said bulbine natalensis is present in an amount therapeutically effective for preventing or treating a disorder or disease state with low testosterone, testosterone deficiency or testosterone insufficiency.

17. A method of preventing and treating disorders and diseases associated with low testosterone, testosterone deficiency or testosterone

18. insufficiency, comprising topically administering to a mammal a substantially testosterone-free composition comprising:
(a) at least one bulbine natalensis compound present in a therapeutically effective amount to increase testosterone levels when administered to said mammal; and
(b) a topical transdermal pharmaceutical carrier effective for topical administration of said at least one bulbine natalensis compound to said mammal.

19. The method according to claim 16, wherein said mammal has a blood serum testosterone less than 1,200 ng/dl or a testosterone blood serum level lower than the normal range for said mammal.

20. The method according to claim 16, wherein bulbine natalensis compound is present in composition an amount of about 0.1 wt/wt % to about 98 wt/wt %.

21. The method according to claim 16, wherein topical administration of said bulbine natalensis compound to a mammal results in increased testosterone blood serum blood levels.

22. The method according to claim 16, wherein topical administration of said bulbine natalensis compound results in a serum testosterone blood serum level greater than 7 ng/dl for males and females.

23. A method according to claim 16, wherein said composition is administered in a single or multiple applications.

24-40. (canceled)
